(19) United States
(12) Patent Application Publication
(10) Pub. No.: US 2013/0256316 A1
(43) Pub. Date: Oct. 3, 2013

(54) Device for Introducing an Aroma into a Carrier Gas Stream

(71) Applicant: Air Liquide, Societe Anonyme pour Etude et Exploitation des Procedes Georges Claude, (US)

(72) Inventor: Britta GLOGAU, Houston, TX (US)

(21) Appl. No.: 13/796,691

(22) Filed: Mar. 12, 2013

(30) Foreign Application Priority Data
Mar. 12, 2012 (EP) EP 12159092.1

(51) Int. Cl. A61L 9/12; B65B 31/02; F17C 1/00

(52) U.S. Cl. CPC A61L 9/12; F17C 1/00; B65B 31/02
USPC 220/583; 53/432; 222/3; 366/107

(57) ABSTRACT

The present invention relates to a device and a method for introducing an aroma into a carrier gas stream. The device has a first connection for a compressed-gas container which stores the carrier gas, at least one second connection for an aroma storage unit and an outlet for a gas mixture. The first connection for a compressed-gas container is flow-connected to the outlet via a first line and the at least one second connection for the aroma storage unit is flow-connected to the outlet via a second line. The second connection for the aroma storage unit is a quick-action coupling connection.

The present invention allows the intuitive operation of a device for introducing an aroma into a carrier gas and also the simple exchange of an aroma storage unit.

### CROSS-REFERENCE TO RELATED APPLICATIONS

This application claims the benefit of priority under 35 U.S.C. §119 (a) and (b) to European Patent Application No. 12159092.1, filed Mar. 12, 2012, the entire contents of which are incorporated herein by reference.

### BACKGROUND

1. Field of the Invention

The present invention relates to a device and a method for introducing an aroma into a carrier gas stream.

2. Related Art

It is known to attract consumers to products so that they notice these by smell. Thus, on the one hand, spaces accessible to the public are subjected to odorous aromas in order to disseminate an agreeable fragrance or draw attention to special products. On the other hand, aromatics are administered to packs of foodstuffs so that, when the pack is opened, a pleasant fresh odour is disseminated.

In all these applications, it is necessary for a carrier gas to be mixed with an aroma. Thus, on the one hand, it is known that the carrier gas is stored together with the aroma gas. On the other hand, it is known to add the aroma to the carrier gas when the aroma is to be administered in doses at the desired location.

On account of the multiplicity of known aromas which can be used for these applications, it is desirable that a carrier gas can be combined with various aromas in a simple way.

It would be desirable to offer aroma-containing gases where the aroma and the gas are premixed and stored in a compressed gas cylinder. However, storage of such an aroma-containing gas in a compressed gas cylinder is disadvantageous due to the tendency of the inner lining of the compressed gas cylinder to adsorb amounts of the aroma. As it is well known that minute amounts of gases can be adsorbed to the inner lining of a compressed gas cylinder and remain adsorbed upon depletion of the cylinder, once a cylinder containing the aroma and gas is depleted, aroma adsorbed to the inner lining of the cylinder would be difficult to remove without requiring special treatment. Such a special treatment would be considered complicated and expensive and consume an undesirable amount of time. Upon refilling of such a depleted cylinder, the aroma would desorb into the newly filled gas, thereby preventing use of the cylinder for containing gases without aromas.

On account of this tendency of aromas to become adsorbed on the inner linings of pressurized gas cylinders, it is desirable that a carrier gas can be combined with various aromas in way that avoids this problem.

### SUMMARY OF THE INVENTION

The object of the present invention, therefore, is to solve at least partially the problems outlined with regard to the prior art. In particular, the object of the present invention is to specify a device and a method, by means of which, by simple handling, various aromas can be fed into a carrier gas.

These objects are achieved by means of a device and a method according to the independent claims. Advantageous developments are specified in the dependent claims and in the description.

The object is achieved by means of a device for introducing an aroma into a carrier gas stream, the device having a connection for a compressed-gas container which stores a carrier gas, at least one connection for an aroma storage unit and an outlet for a gas mixture, the connection for the compressed-gas container being flow-connected to the outlet via a first line and the at least one connection for the aroma storage unit being flow-connected to the outflow via a second line, the connection for the aroma storage unit being a quick-action coupling connection.

The object is also achieved by way of a device for introducing an aroma into a carrier gas stream. The device comprises: at least one first connection adapted and configured to connect the device to a source or sources of pressurized carrier gas; a first line fluidly communicating with the at least one first connection; at least one second connection adapted and configured to connect the device to an aroma storage unit; a second line fluidly communicating with the at least one second connection; a third line fluidly communicating with the first and second lines and having an outlet through which a combined flow of aroma and pressurized carrier gas flows. The first and second lines feed flows of pressurized carrier gas and aroma into the third line, respectively, and each one of the at least one second connection is a quick-action coupling connection.

The object is also achieved by way of an aroma delivery system, comprising an aroma storage unit containing aroma that is connected to one of the above-described devices via one of the at least one second connection and a source of pressurized carrier gas also connected to the above device via one of the at least one first connection.

The object is also achieved by way of a method for introducing an aroma into a carrier gas stream using an introduction device, the aroma being stored in an aroma storage unit, the introduction device having a first connection, a second connection, a third line leading to an outlet, a first line in fluid communication with the third line and extending between the first connection and the third line, and a second line in fluid communication with the third line and extending between the second connection and the third line. The method comprises the following steps. The aroma storage unit is connected to the introduction device via the second connection, the second connection being a quick-action coupling connection. The carrier gas is allowed to flow out of a source of the carrier gas and into the introduction device via the first connection such that the carrier gas carries aroma into the third line and out of an outlet of the introduction device.

Any of the above devices, the delivery system or the method may include one or more of the following aspects:
each of the at least one first connection is threaded to receive a correspondingly threaded connector from a source of the pressurized carrier gas.
the source of the pressurized carrier gas is a compressed gas cylinder.
the source of the pressurized carrier gas is a vaporizer fluidly communicating with a container of liquefied carrier gas.
the device is adapted and configured to be connected unreleasably to the compressed-gas container.
the device further comprises a shut-off valve disposed in each of the first and second line and a switch, each of the shut-off valves being actuable via the switch.
the device further comprises a pressure reduction valve disposed in the second line that is adapted and configured to allow the pressure to be reduced in steps to discrete levels.
the device further comprises a pressure reduction disposed in the second line that is adapted and configured to allow the pressure to be reduced in a continuous manner.
the device further comprises a first filling-level indicator that is adapted and configured to indicate a fill level of the carrier gas in the cylinder.
the device further comprises a protective cap enclosing the first and second lines.
the device further comprises a handle attached to the protective cap.
the device further comprises a silencer disposed in the first line and/or the second line.
the aroma storage unit includes a pierceable elastomeric septum that seals the aroma in the storage unit when the aroma storage unit is disconnected from the device.
the aroma storage unit includes a calibrated orifice
the aroma storage container includes a pressure indicator indicative a pressure inside the storage unit.
the device further comprises one or more additional connections fluidly communicating with the third line, each of the one or more additional connections being adapted and configured to be to connected to the additional sources of pressurized carrier gas.
the device further comprises one or more additional connections fluidly communicating with the third line, each of the one or more additional connections is connected to the additional sources of pressurized carrier gas and the additional sources of pressurized gas are compressed gas cylinders.
the source of the pressurized carrier gas is a compressed gas cylinder.
the compressed gas cylinder is connected to the second connection via a cylinder valve with an integrated pressure regulator (VIPR).
the source of the pressurized carrier gas is an evaporator fluidly communicating with a container of liquefied carrier gas.
the flow of carrier gas and aroma is introduced into an interior of food packaging.
the flow of carrier gas and aroma is introduced into a space used by people.
the space is selected from the group consisting of an interior of a hired vehicle, an interior of public transportation, an interior of a public restroom, public spaces, an interior of a hotel, an interior of a lobby, an interior of a retail store, an interior of a bakery, an interior of a coffee shop, and an interior of an airport.

Aroma is understood in the context of the present invention to mean not only the aroma itself, but also an aroma which is dissolved in a gas, aroma which is dissolved in a liquid, and aroma which is introduced in gas form into the carrier stream. What may be considered as carrier gas are all gases and mixtures of at least two of these gases, such as, for example, compressed air, oxygen, nitrogen, helium, xenon, argon, krypton, hydrogen, CO₂ (carbon dioxide). Typically, the gas is compressed air or CO₂-enriched air having a CO₂ concentration of less than 5,000 ppm. The gases may also be any ones typically used in the medical field or in the food industry. For example, the carrier gas may be a Modified Atmosphere Gas (MAP) conventionally used with packaged foods.

The aromas may be added in pure or mixed form. Addition may take place in solid form, as a liquid, as a gas or in a dissolved form, adsorbed form, or absorbed form. The aromas may be stored under pressure in liquid and/or gaseous form in a disposable or reusable cartridge. When the aromas are present in gaseous form, typically the gas mixed with the aroma in the aroma storage unit is nitrogen, helium, xenon, argon, krypton, hydrogen, carbon dioxide, or mixtures of at least two of these gases, such as, for example, compressed air.

A connection is understood to be an element which makes mechanical connection between the device and the compressed-gas container or aroma storage unit and, on the other hand, ensures a flow connection of the first line and of the second line to the compressed-gas container and to the aroma storage unit respectively. Thus, in a preferred embodiment, the connection for the compressed-gas container is an internal thread which can be screwed onto an external thread and the compressed-gas container and through which the carrier gas flows centrally during operation. An outlet is to be interpreted as being that portion of a line out of which the carrier gas, together with the introduced aroma, emerges from the device and is led on for further use. Thus, the first line and the second line may end jointly in the connection, but it is preferable that the first line and the second line are converged and the line thus arising as a third line leads to the outlet.

The aroma storage unit preferably comprises a container in which the aroma is stored, if appropriate in a solution. The aroma storage unit has a quick-action coupling connection so that the container can be flow-connected to the second line.

Quick-action coupling connections are understood to mean connection devices which can be actuated, without an additional tool, only with the hands, preferably only with one hand. The quick-action coupling connection on the device may therefore be, in particular, a plug or a recess which are plugged one into the other. After the counter pieces have been brought together, they are latched with one another. This latching can be released by means of a simple manipulation. In particular, the release of the latched connection takes place by the action of force upon the quick-action coupling in or opposition to the coupling direction. A quick-action coupling generates a mechanical connection which opposes the internal pressure prevailing during operation.

On account of the quick-action coupling connection, it is possible to exchange the aroma storage unit intuitively in a simple and quick way. This, on the one hand, makes it possible to use different aromas in one place of use and, on the other hand, the aroma storage units can thus be designed for specific applications. It is therefore preferable that the aroma storage units have reusable elements. However, it is also possible that the aroma storage unit is designed as a disposable product.

According to an advantageous development of the invention, the device is connected unreleasably to the compressed-gas container. An unreleasable connection is to be understood as meaning a connection which cannot be separated without the action of force and without destroying the device. The unreleasable connection of the compressed-gas container to the device ensures intuitive and simple operation. Thus, the only component which can be separated from the device is the aroma storage unit which can be exchanged, as required. During operation, the device is connected via the outlet to a line for delivering the carrier gas mixed with aroma to a nozzle. When the contents of the compressed-gas container, that is to say the carrier gas, have been consumed, the compressed-gas container and the entire device are exchanged. This also ensures that the device can be regularly maintained and checked, to be precise, when the compressed-gas container is being exchanged.

According to yet another advantageous refinement of the invention, a shut-off valve is arranged in each case in the first line and in the second line, and these shut-off valves can preferably be actuated simultaneously via a common switch. By the common switch being actuated, both the carrier gas stream and the flow out of the aroma storage unit can be simultaneously interrupted or restored, without further state variables of the flows being influenced directly. Thus, the introduction of aroma into the carrier gas stream can be started and ended, in particular, without any variation in the mix ratio of the carrier stream and aroma.

According to yet a further development of the device, a pressure-reducing valve or a flow reducer is arranged in the second line for the stepped reduction of the flow in stipulated steps. By means of the pressure-reducing valve, the flow of the aroma and therefore its concentration in the mixture of carrier gas and aroma can be set continuously. In particular, the pressure-reducing valve is independent of the shut-off valve. In contrast, by means of the flow reducer, the flow can be metered in steps (i.e., the flow can be changed in step-wise manner to any one discrete flow rate). As one example, the flow may be metered in 2, 4, 6 or 8 steps, in which case a certain concentration of aroma in the carrier gas can be assigned to each step. This stepped reduction of the aroma gas flow also takes place independently of the shut-off valve.

It is advantageous, moreover, if the device comprises at least one of the following elements:
a filling-level indicator for the compressed-gas containers and
a protective cap, in particular with a carrying grip.

By means of the filling-level indicator, it can be determined in a simple way when the compressed-gas container will be empty and when, if appropriate, a new compressed-gas container has to be attached. The protective cap protects the device from the action of mechanical force.

It is advantageous, moreover, if a silencer is integrated in the first line and/or in the second line. A silencer ensures, precisely at quiet locations, that the device does not cause any noise nuisance during operation.

According to a further aspect, an aroma storage unit for connection to a device according to the invention is proposed, which comprises a container, preferably a compressed-gas container, and at least one of the following elements:
a self-sealing element,
a calibrated orifice,
a tension-based filling-level indicator,
a quick-action coupling connection.

The calibrated orifice ensures that, after an aroma storage unit has been exchanged, the same quantity of aroma flows into the device as before the exchange, provided that the settings on the device are the same. The quick-action coupling connection ensures connection to the device according to the invention. The tension, measured by measuring the mechanical tension on the outer casing of the container, makes it possible in a simple way to indicate the quantity of aroma remaining in the container. The self-sealing element, in turn, ensures a gas-tight connection of the container to the device according to the invention via the quick-action coupling connection, in that, during the operation of closing the quick-action coupling, a sealing material is pressed against a corresponding sealing surface. An example of a tension-based filling-level indicator is a strain gauge.

According to a further aspect of the invention, a method for introducing an aroma into a carrier gas stream is proposed, the aroma being stored in an aroma storage unit and a carrier gas being stored in a compressed-gas container, comprising at least the following steps:
connection of the aroma storage unit to an introduction device,
leading of the carrier gas stream out of the compressed-gas container through the introduction device,
introduction of the aroma out of the aroma storage unit into the carrier gas stream in the introduction device, the introduction device being connected to the aroma storage unit in a quickly releasable way.

The introduction device is, in particular, a device according to the invention. On account of this, however, it is precisely the device according to the invention which is suitable for carrying out the method according to the invention. The features and advantages disclosed with regard to the device according to the invention can be transferred and applied to the method according to the invention, and vice-versa.

The aroma storage unit is thus connected to the device according to the invention via the quick-action coupling connection and, in particular thereafter, the carrier gas stream is led through the introduction device, at the same time introduction of the aroma into the introduction device and therefore into the carrier gas stream also taking place.

It is preferable that the gas stream containing aroma is introduced into a pack for a foodstuff. In particular, the gas stream containing aroma is introduced into a pack of ice, meat, sweets, snacks or drinks. However, introduction may also take place in a pack of cosmetics. On account of this, however, the use of the device according to the invention for introducing aroma gases into corresponding packs is also proposed.

Alternatively, it is also preferable that the gas stream containing aroma is introduced into a space used by people. In particular, the gas containing aroma may be introduced into a hired vehicle, public transport means, public toilet, public spaces, hotels, lobbies, boutiques, bakeries, coffee shops or airports. On account of this, however, it is also proposed precisely to use a device according to the invention for introducing aromatized gases into such a space used by people.

In the case where the aroma storage unit having a weaker aroma is replaced with an aroma storage unit having a stronger aroma, the device may include additional connections each of which is adapted and configured to connect to additional sources of carrier gas (such as a compressed gas cylinder). For example, carrier gas from a first source of a carrier gas carries aroma out of the aroma storage device and into the first line. The carrier gas from the first source carries the aroma into the second line. An additional source of carrier gas is connected to the third line downstream of where the first line meets the second line. This additional carrier gas dilutes the already-combined aroma/carrier gas to dilute the stronger aroma to an acceptable level. Such serial dilution of the aroma can be carried out by as many different sources of carrier gas as needed. Alternatively, the pressure of the carrier gas in the first line may be increased to a level sufficient to dilute the aroma in order to achieve the acceptable level of aroma.

The invention is explained in more detail by way of example by means of the present drawing, without being restricted to the exemplary embodiments shown there.

### BRIEF DESCRIPTION OF THE FIGURES

FIG. 1 shows diagrammatically a device for introducing an aroma into a carrier gas stream.

### DETAILED DESCRIPTION OF THE INVENTION

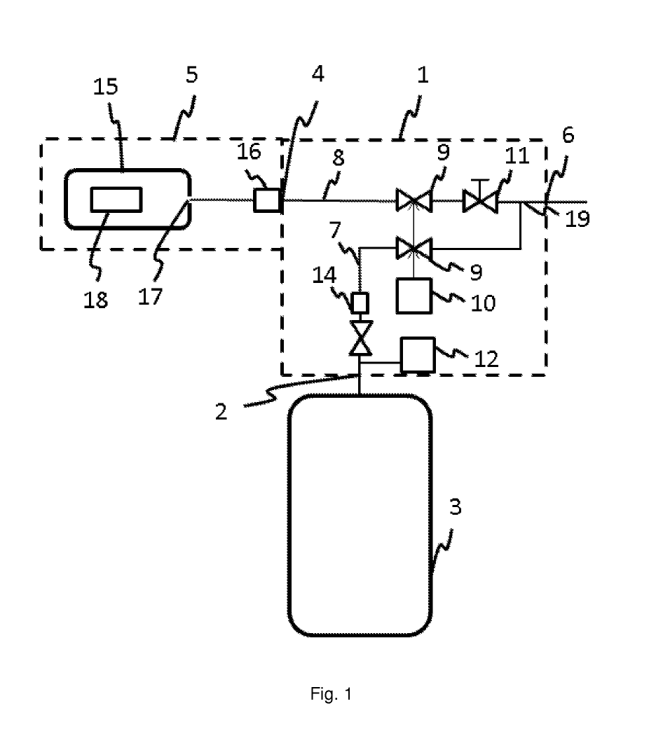

FIG. 1 shows a device 1 to which a compressed-gas container 3 and an aroma storage unit 5 are connected. The device 1 has a first connection 2, by which the device 1 is connected to the compressed-gas container 3. A carrier gas is stored in the compressed-gas container 3. The device 1 has, furthermore, a quick-action coupling connection 4, via which the device 1 is connected to the aroma storage unit 5. In the device 1, a first line 7 is flow-connected to the first connection 2 and a second line 8 is flow-connected to the quick-action coupling connection 4. The first line 7 and the second line 8 are converged in the device 1 and are led further on as a third line 19 to an outlet 6. The device 1 is connected to a spray nozzle, not illustrated, via the outlet 6.

A shut-off valve 9, which can be actuated via a common switch 10, is formed in each case both in the first line 7 and in the second line 8. Furthermore, a pressure-reducing valve 11 is formed in the second line 8 and serves for the continuous or stepped reduction of the flow in the second line 8. A silencer is formed in the first line 7 and reduces the possible generation of noise when the carrier gas is conducted through the first line 7.

The aroma storage unit 5 comprises a container 15 which has a calibrated orifice 17 and a pressure sensor 18 indicating a filling-level of gas in the unit 5. Moreover, the aroma storage unit 5 has a quick-action coupling connection 4 which is provided with a self-sealing element 16 and via which the aroma storage unit 5 is flow-connected mechanically to the device 1. The self-sealing element may be a pierceable elastomeric septum which seals the aroma in the unit 5 when the unit 5 is disconnected from the device 1.

During operation, the carrier gas is conducted out of the compressed-gas container 3 via the first connection 2 and through the first line 7 to the outlet 6. In one way of carrying out the invention, the pressure of the carrier gas in the compressed gas cylinder 3 is higher than the pressure in the second line 8, which in turn, is higher than the pressure in the third line 19. Aromatics, which may be stored in a container 15 in a gaseous solution, in liquid solution, or adsorbed or absorbed on a solid, are introduced into the carrier gas via the quick-action coupling connection 4 and through the second line 8. The mixture of carrier gas and aroma arrives at the outlet, from where the mixture is led to a suitable nozzle for the respective application. The quantity of aroma to be added can be set via the continuously or stepwise adjustable pressure-reducing valve 11. The two shut-off valves 9 can be actuated via the switch 10, so that the delivery of both carrier gas from the compressed-gas container 3 and of aroma from the container 15 can be simultaneously stopped or started. In this case, in particular, the pressure-reducing valve 11 does not have to be actuated, and therefore the same conditions prevail after introduction has been restarted. Since a quick-action coupling connection 4 is provided between the aroma storage unit 5 and the device 1, when the aroma is consumed or is changed a simple exchange of the aroma storage unit 5 can take place. Moreover, the device 1 can be connected unreleasably (for example, by welding) to the compressed gas cylinder 3, so that, when the carrier gas is consumed, the complete unit consisting of the device 1 and of the compressed-gas container 3 is exchanged. This ensures, in particular, that the device 1 is regularly maintained. Alternatively, the device 1 can be connected unreleasably (for example, by welding) to a cylinder valve and integrated pressure regulator (VIPR) which is attached to the compressed gas cylinder 3, so that, when the carrier gas is consumed, the complete unit consisting of the device 1 and the VIPR are exchanged with a supplier with or without the compressed-gas container 3. Again, this ensures, in particular, that the device 1 is regularly maintained. The present invention allows the intuitive operation of a device 1 for introducing an aroma into a carrier gas and also the simple exchange of an aroma storage unit 5.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

### LIST OF REFERENCE SYMBOLS

1 Device

2 First connection

3 Compressed-gas container

4 Quick-action coupling connection

5 Aroma storage unit

6 Outlet

7 First line

8 Second line

9 Shut-off valve

10 Switch

11 Pressure-reducing valve

12 First filling-level indicator

13 Protective cap

14 Silencer

15 Container

16 Self-sealing element

17 Calibrated orifice

18 Second filling-level indicator

19 Third line

What is claimed is:

1. A device for introducing an aroma into a carrier gas stream, comprising:
a first connection adapted and configured to connect the device to a source or sources of pressurized carrier gas;
a first line fluidly communicating with the first connection;
at least one second connection adapted and configured to connect the device to an aroma storage unit;
a second line fluidly communicating with the at least one second connection;
a third line fluidly communicating with the first and second lines and having an outlet through which a combined flow of aroma and pressurized carrier gas flows, wherein the first and second lines feed flows of pressurized carrier gas and aroma into the third line, respectively, and each one of the at least one second connection is a quick-action coupling connection.

2. The device of claim 1, wherein each of the first connection is threaded to receive a correspondingly threaded connector from a source of the pressurized carrier gas.

3. The device of claim 2, wherein the source of the pressurized carrier gas is a compressed gas cylinder.

4. The device of claim 2, wherein the source of the pressurized carrier gas is a vaporizer fluidly communicating with a container of liquefied carrier gas.

5. The device of claim 1, wherein the device is adapted and configured to be connected unreleasably to the compressed-gas container.

6. The device of claim 1, further comprising:
a shut-off valve disposed in each of the first and second line; and
a switch, each of the shut-off valves being actuable via the switch.

7. The device of claim 1, further comprising a pressure reduction valve disposed in the second line or third line that is adapted and configured to allow the pressure to be reduced in steps to discrete levels.

8. The device of claim 1, further comprising a pressure reduction disposed in the second line or third line that is adapted and configured to allow the pressure to be reduced in a continuous manner.

9. The device of claim 3, further comprising a first filling-level indicator that is adapted and configured to indicate a fill level of the carrier gas in the cylinder.

10. The device of claim 3, further comprising a protective cap enclosing the first and second lines.

11. The device of claim 10, further comprising a handle attached to the protective cap.

12. The device of claim 1, further comprising a silencer disposed in the first line and/or the second line.

13. The device of claim 1, further comprising one or more additional connections fluidly communicating with the third line, each of the one or more additional connections being adapted and configured to be connected to the additional sources of pressurized carrier gas.

14. An aroma delivery system, comprising an aroma storage unit containing aroma that is connected to the device of claim 1 via one of the at least one second connection and a source of pressurized carrier gas connected to the device of claim 1 via one of the at least one first connection.

15. The aroma delivery system of claim 14, wherein the aroma storage unit includes a pierceable elastomeric septum that seals the aroma in the storage unit when the aroma storage unit is disconnected from the device of claim 1.

16. The aroma delivery system of claim 14, wherein the aroma storage unit includes a calibrated orifice

17. The aroma delivery system of claim 14, wherein the aroma storage container includes a pressure indicator indicative a pressure inside the storage unit.

18. The aroma delivery system of claim 14, wherein the source of the pressurized carrier gas is a compressed gas cylinder.

19. The aroma delivery system of claim 18, wherein the compressed gas cylinder is connected to the second connection via a cylinder valve with an integrated pressure regulator (VIPR).

20. The aroma delivery system of claim 14, wherein the source of the pressurized carrier gas is an evaporator fluidly communicating with a container of liquefied carrier gas.

21. The aroma delivery system of claim 14, further comprising:
one or more additional connections fluidly communicating with the third line, each of the one or more additional connections; and
a corresponding number of additional sources of pressurized gas connected to the additional connections, wherein the additional sources of pressurized gas are compressed gas cylinders.

22. A method for introducing an aroma into a carrier gas stream using an introduction device, the aroma being stored in an aroma storage unit, the introduction device having a first connection, a second connection, a third line leading to an outlet, a first line in fluid communication with the third line and extending between the first connection and the third line, and a second line in fluid communication with the third line and extending between the second connection and the third line, comprising the steps of:
connecting the aroma storage unit to the introduction device via the second connection, the second connection being a quick-action coupling connection;
allowing the carrier gas to flow out of a source of the carrier gas and into the introduction device via the first connection such that the carrier gas carries aroma into the third line and out of an outlet of the introduction device.

23. The method of claim 22, wherein the flow of carrier gas and aroma is introduced into an interior of food packaging.

24. The method of claim 22, wherein the flow of carrier gas and aroma is introduced into a space used by people.

25. The method of claim 22, wherein the space is selected from the group consisting of an interior of a hired vehicle, an interior of public transportation, an interior of a public restroom, public spaces, an interior of a hotel, an interior of a lobby, an interior of a retail store, an interior of a bakery, an interior of a coffee shop, and an interior of an airport.
